(19) United States
(12) Patent Application Publication
(10) Pub. No.: US 2024/0256723 A1
(43) Pub. Date: Aug. 1, 2024

(54) SYSTEMS AND METHODS FOR CLINICAL WORKSPACE SIMULATION

(71) Applicant: Covidien LP, Mansfield, MA (US)

(72) Inventors: Max L. Balter, Boston, MA (US); Michael A. Eiden, Somerville, MA (US); William J. Peine, Ashland, MA (US); Unnas W. Hussain, Boston, MA (US); Justin R. Chen, Morganville, NJ (US)

(21) Appl. No.: 18/289,609

(22) PCT Filed: May 18, 2022
(86) PCT No.: PCT/US2022/029737

§ 371 Date: Nov. 6, 2023

Related U.S. Application Data
(60) Provisional application No. 63/196,997, filed on Jun. 4, 2021.

(51) Int. Cl. G06F 30/13

(52) U.S. Cl. CPC G06F 30/13

(57) ABSTRACT

A computer-implemented method for clinical workplace simulation includes capturing a surgical parameter from one or more robotic surgical operations, based on a sensor; and determining an optimized surgical parameter based on the captured surgical parameter. The surgical parameter includes a patient habitus, a port location in a first patient, and/or a robotic arm placement relative to the first patient. The optimized surgical parameter includes an optimized port placement location in a second patient, and/or an optimized robotic arm placement location relative to the second patient.

### CROSS-REFERENCE TO RELATED APPLICATIONS

The present application claims the benefit of the filing date of provisional U.S. Patent Application No. 63/196,997, filed Jun. 4, 2021.

### BACKGROUND

### Technical Field

The disclosure generally relates to systems and methods for clinical workspace simulations. In particular, the present disclosure is directed to a simulated setup of surgical robotic systems.

### Background of Related Art

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a worksite within the patient's body.

Setup time for robotic surgical systems can be lengthy and cumbersome, and may not account for potential collisions between robotic arms during a surgery. Thus, there is a need for systems for virtual placement of surgical robotic system to determine initial robotic system component placement.

### SUMMARY

In accordance with aspects of the disclosure, a computer-implemented method for clinical workspace simulation is presented. The method includes receiving a first input from a user indicating an area for a virtual operating room, receiving a second input from the user selecting at least one virtual object, rendering the virtual operating room based on the received input indicating the area for the virtual operating room, rendering the at least one virtual object relative to a virtual surgical table in the virtual operating room, and displaying on a display the rendered virtual operating room and the at least one virtual object relative to a virtual surgical table in the virtual operating room.

In an aspect of the disclosure, the virtual object may include a virtual robotic arm, a virtual surgical tower, and/or a virtual surgical console.

In another aspect of the disclosure, the display may include a display of at least one of a mobile device, a computer, or a virtual reality headset.

In yet another aspect of the disclosure, the displayed virtual operating room may include a viewpoint. The viewpoint includes at least one of a perspective or a zoom level.

In a further aspect of the disclosure, the method may further include receiving a third input indicating a change in the viewpoint and displaying the changed viewpoint.

In another aspect of the disclosure, the method may further include receiving a fourth input for interacting with the virtual object.

In yet another aspect of the disclosure, the method may further include displaying a text window indicating information relating to the virtual object.

In a further aspect of the disclosure, the method may further include displaying an animation of the virtual object.

In yet a further aspect of the disclosure, the method may further include displaying an animation of the virtual object.

In another aspect of the disclosure, the method may further include generating a virtual patient, including a simulated patient habitus based on at least one of a fifth input or a patient medical record.

In another aspect of the disclosure, the method may further include receiving a sixth input placing the at least one virtual object at a virtual object placement location and rendering the at least one virtual object at the virtual object placement location.

In yet another aspect of the disclosure, the method may further include receiving an indicated anatomy of the virtual patient. The virtual object placement location may be further based on targeting the indicated anatomy.

In accordance with aspects of the disclosure, a computer-implemented method for clinical workspace simulation is presented. The method includes capturing a surgical parameter from one or more robotic surgical operations, based on a sensor; and determining an optimized surgical parameter based on the captured surgical parameter. The surgical parameter includes a patient habitus, a port location in a first patient, and/or a robotic arm placement relative to the first patient. The optimized surgical parameter includes an optimized port placement location in a second patient, and/or an optimized robotic arm placement location relative to the second patient.

In an aspect of the disclosure, the method may further include generating a simulated patient including a simulated patient habitus based on a first user input and/or a patient medical record.

In another aspect of the disclosure, the method may further include receiving a second user input for modifying the simulated patient habitus and modifying the simulated patient habitus based on the user input.

In yet another aspect of the disclosure, the method may further include generating a setup guide based on the generated simulated patient habitus and the determined optimized surgical parameter and displaying the setup guide on a display. The setup guide may include the optimized surgical port placement location and/or the optimized robotic arm placement location.

In a further aspect of the disclosure, generating the setup guide may include generating a revised surgical port placement location in a trunk segment of the simulated patient.

In a further aspect of the disclosure, the revised surgical port placement location may be further based on the simulated patient habitus.

In an aspect of the disclosure, the surgical port entry point may be further based on a body habitus of the patient.

In yet a further aspect of the disclosure, the method may further include generating a revised optimized robotic arm placement location relative to the virtual patient.

In yet a further aspect of the disclosure, the method may further include receiving an indicated anatomy from a user. The optimized robotic arm placement location may be further based on targeting the indicated anatomy.

In another aspect of the disclosure, the optimized robotic arm placement location may include a first location for a first robotic arm and a second location for a second robotic arm. The method may further include determining a risk of collision between the first robotic arm and a second robotic arm, based on the first location, the second location, the optimized surgical port placement location, and/or the simulated patient habitus; and displaying a warning of the risk of collision on the display.

In accordance with aspects of the disclosure, a system for clinical workspace simulation includes a sensor configured to sense a surgical parameter, a processor; and a memory. The memory includes instructions stored thereon, which, when executed by the processor, cause the system to capture the surgical parameter from one or more robotic surgical operations, based on the sensor; and determine an optimized surgical parameter based on the captured surgical parameter. The surgical parameter includes a patient habitus, a surgical port location in a first patient, and/or a robotic arm placement relative to the first patient. The optimized surgical parameter includes an optimized surgical port placement location in a second patient and/or an optimized robotic arm placement location relative to the second patient.

In yet a further aspect of the disclosure, the instructions, when executed by the processor, may further cause the system to generate a simulated patient, including a simulated patient habitus based on a first user input and/or a patient medical record.

In yet another aspect of the disclosure, the instructions, when executed by the processor, may further cause the system to receive a second user input for modifying the simulated patient habitus and modify the simulated patient habitus based on the user input.

In a further aspect of the disclosure, the instructions, when executed by the processor, may further cause the system to generate a setup guide based on the generated simulated patient habitus and the determined optimized surgical parameter and display the setup guide on a display. The setup guide includes the optimized surgical port placement location and/or the optimized robotic arm placement location.

In yet a further aspect of the disclosure, when generating the setup guide, the instructions, when executed by the processor, may further cause the system to generate a revised surgical port placement location in a trunk segment of the simulated patient.

In an aspect of the disclosure, the revised surgical port placement location may be further based on the simulated patient habitus.

In another aspect of the disclosure, when generating the setup guide, the instructions, when executed by the processor, may further cause the system to generate a revised optimized robotic arm placement location relative to the virtual patient.

In yet another aspect of the disclosure, the optimized robotic arm placement location may be further based on the simulated patient habitus.

In a further aspect of the disclosure, the optimized robotic arm placement location includes a first location for a first robotic arm and a second location for a second robotic arm. The instructions, when executed by the processor, may further cause the system to determine a risk of collision between the first robotic arm and a second robotic arm, based on the first location, the second location, the optimized surgical port placement location, and/or the simulated patient habitus; and display a warning of the risk of collision on the display.

In accordance with aspects of the disclosure, a non-transitory computer-readable medium storing instructions which, when executed by a processor, cause the processor to perform a method including accessing a surgical parameter from one or more robotic surgical operations, based on a sensor; determining an optimized surgical parameter based on the captured surgical parameter; generating a simulated patient including a simulated patient habitus based on a first user input and/or a patient medical record; generating a setup guide based on the generated simulated patient habitus and the determined optimized surgical parameter. The setup guide includes the optimized surgical port placement location and/or the optimized robotic arm placement location; and displaying the setup guide on a display. The surgical parameter includes a patient habitus, a surgical port location in a first patient, and/or a robotic arm placement relative to the first patient. The optimized surgical parameter includes an optimized surgical port placement location in a second patient and/or an optimized robotic arm placement location relative to the second patient.

### BRIEF DESCRIPTION OF THE DRAWINGS

Various aspects of the disclosure are described herein with reference to the drawings wherein:

FIG. 1 is a schematic illustration of a surgical robotic system including a control tower, a console, and one or more surgical robotic arms according to an aspect of the disclosure;

FIG. 2 is a perspective view of a surgical robotic arm of the surgical robotic system of FIG. 1 according to an aspect of the disclosure;

FIG. 3 is a perspective view of a setup arm with the surgical robotic arm of the surgical robotic system of FIG. 1 according to an aspect of the disclosure;

FIG. 4 is a schematic diagram of a computer architecture of the surgical robotic system of FIG. 1 according to an aspect of the disclosure;

FIG. 5 is a flow chart for a computer-implemented method for clinical workspace simulation according to an aspect of the disclosure;

FIG. 6 is a flow diagram of a machine learning algorithm of the computer-controlled method for clinical workspace simulation using the system of FIG. 1;

FIG. 7 is a diagram of layers of a neural network of FIG. 6 in accordance with aspects of the disclosure;

FIG. 8 is a diagram of a workflow for the computer-implemented method for clinical workspace simulation of FIG. 5, according to an aspect of the disclosure;

FIG. 9 is a diagram of a workflow for the computer-implemented method for clinical workspace simulation of FIG. 5, according to an aspect of the disclosure;

FIG. 10 is an image of a setup guide of the clinical workspace simulation with virtual surgical ports according to an aspect of the disclosure;

FIGS. 11-13 are images of the setup guide showing virtual robotic arms placed relative to a virtual surgical table, according to an aspect of the disclosure;

FIG. 14 is an image of an operating room design screen, according to an aspect of the disclosure;

FIG. 15 is an image of the setup guide showing virtual robotic arms placed relative to a virtual surgical table, according to an aspect of the disclosure; and

FIG. 16 is an image of a virtual generator of a virtual system tower of the virtual operating room of FIG. 15, according to an aspect of the disclosure.

### DETAILED DESCRIPTION

Aspects of the presently disclosed surgical robotic system are described in detail with reference to the drawings, in which like reference numerals designate identical or corresponding elements in each of the several views.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller or on a user device, including, for example, a mobile device, a personal computer, or a server system.

As will be described in detail below, the disclosure is directed to a simulated setup of a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

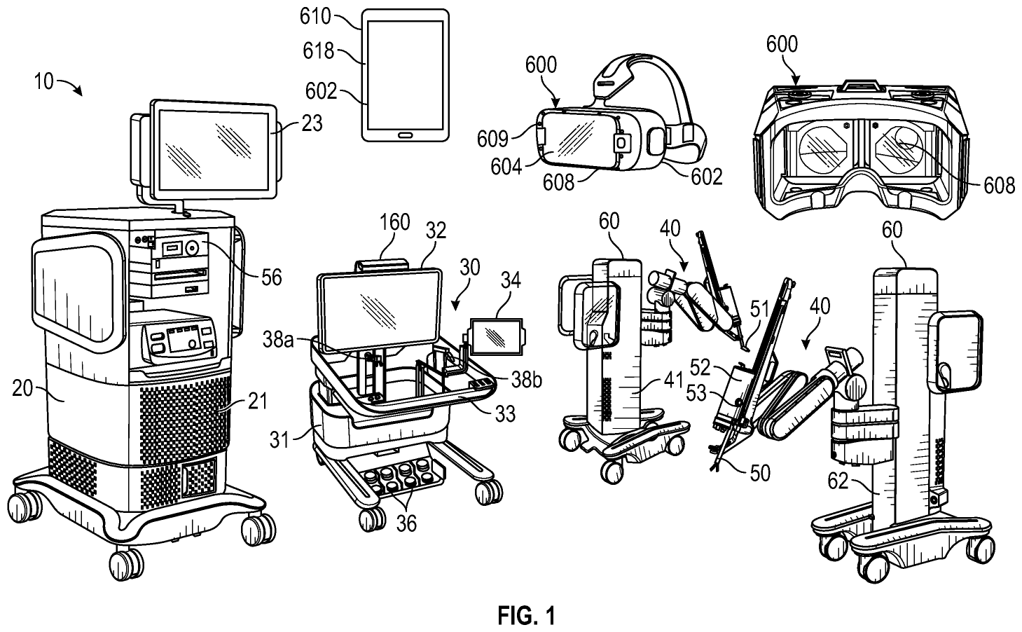

With reference to FIG. 1, a surgical robotic system 10 generally includes a virtual reality headset 600 and/or a mobile device 610 (e.g., a tablet), a control tower 20, which is connected to all of the components of the surgical robotic system 10, including a surgical console 30 and one or more robotic arms 40. Each of the robotic arms 40 includes a surgical instrument 50 removably coupled thereto. Each of the robotic arms 40 is also coupled to a movable cart 60.

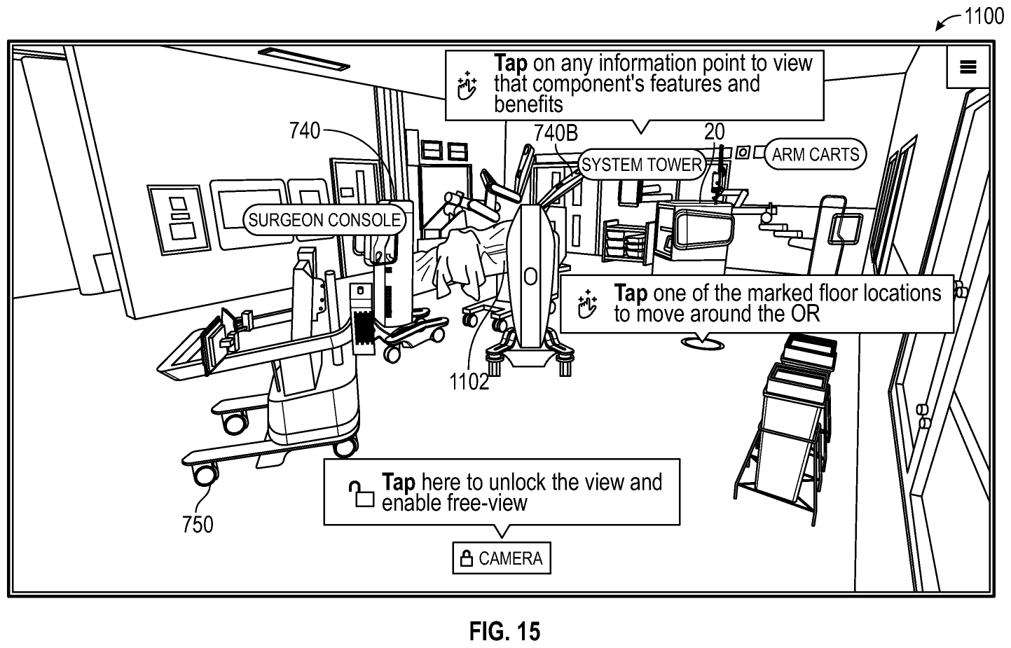

The virtual reality headset 600 configured to display a virtual view generally includes a controller 602, an imaging device 604, and a display 608. The controller 602 includes a memory configured to have instructions stored thereon and a processor configured to execute the instructions. The mobile device 610 configured to display a virtual view generally includes a controller 602, and a display 618. The controller 602 includes a memory configured to have instructions stored thereon and a processor configured to execute the instructions. The mobile device 610 and/or the virtual reality headset 600 may display virtual objects such as a virtual robot arm in a virtual operating room (FIG. 15). For example, the virtual reality headset 600 can provide users advice on how to position various virtual objects to help setup an operating room for a surgery.

The surgical instrument 50 is configured for use during minimally invasive surgical procedures. In aspects, the surgical instrument 50 may be configured for open surgical procedures. In aspects, the surgical instrument 50 may be an endoscope, such as an endoscopic camera 51, configured to provide a video feed for the user. In further aspects, the surgical instrument 50 may be an electrosurgical forceps configured to seal tissue by compressing tissue between jaw members and applying electrosurgical current thereto. In yet further aspects, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

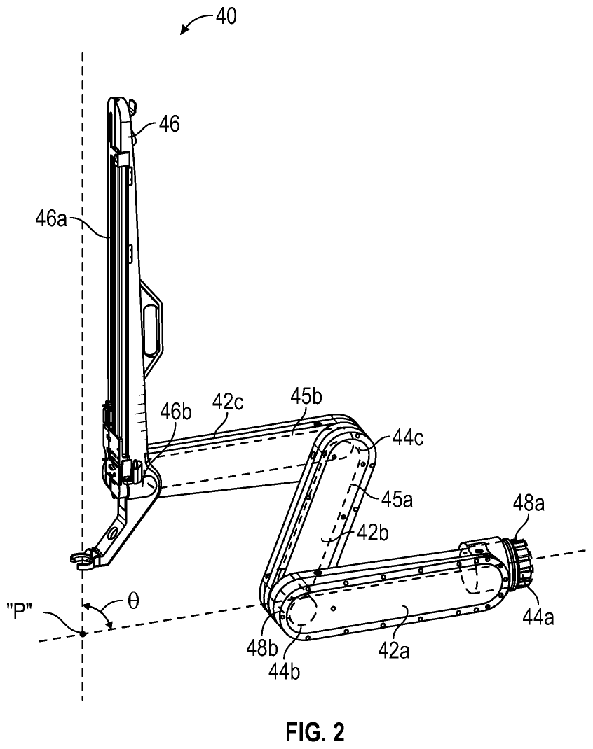

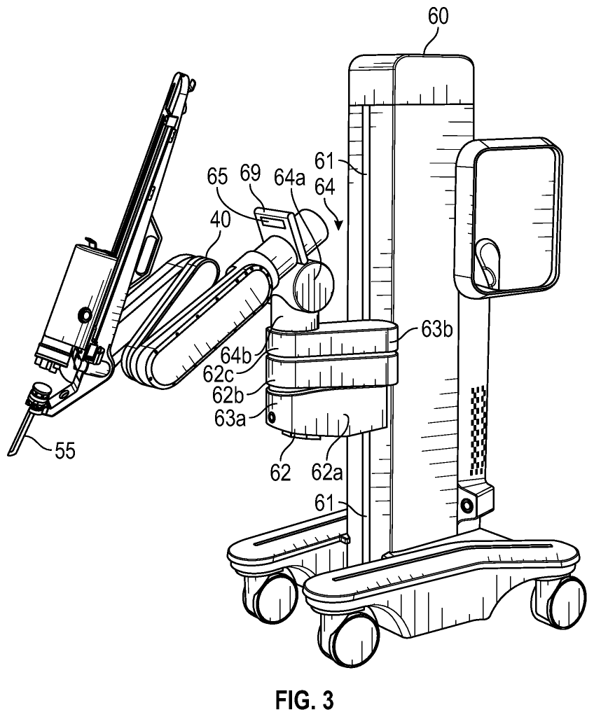

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

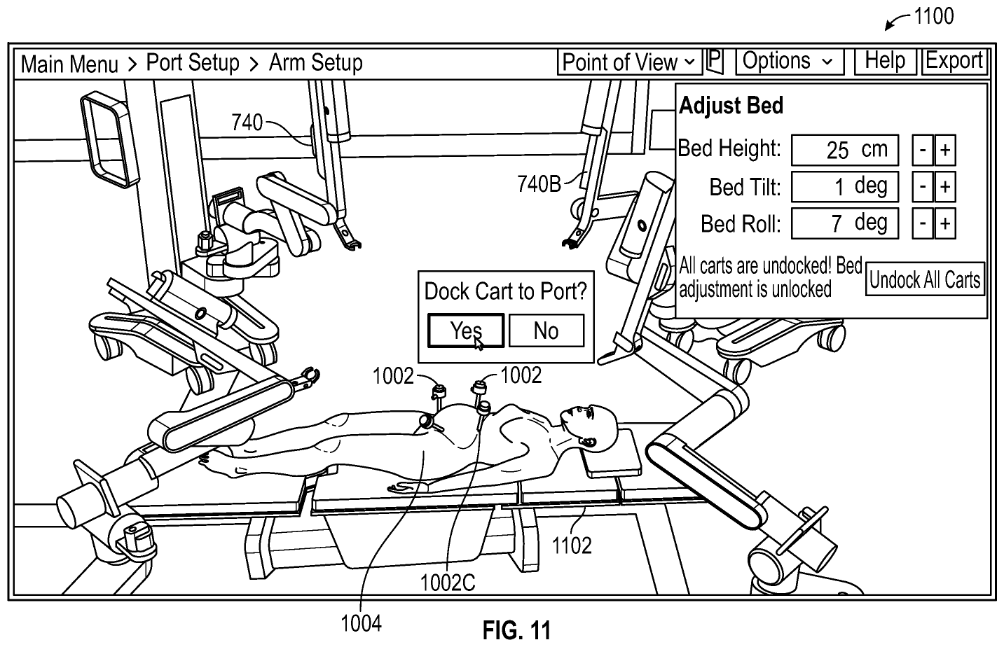

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table 1102 (FIG. 11). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In aspects, some, or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

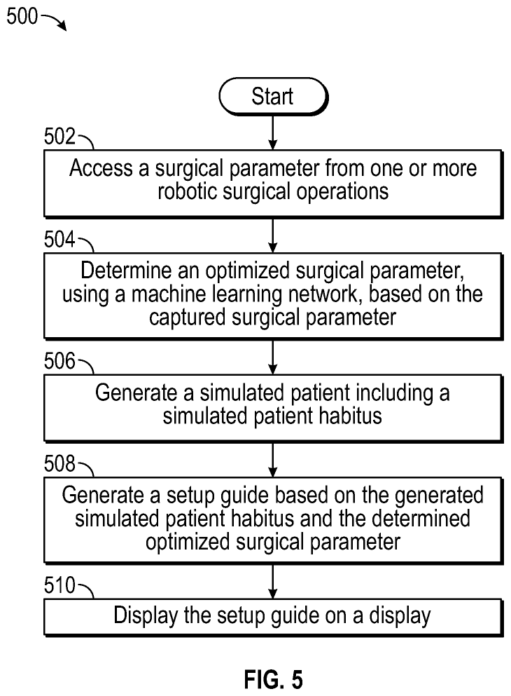

The robotic arm 40 also includes a plurality of manual override buttons 53 (FIGS. 1 and 5) disposed on the IDU 52 and the setup arm 62, which may be used in a manual mode. The user may press one or more of the buttons 53 to move the component associated with the button 53.

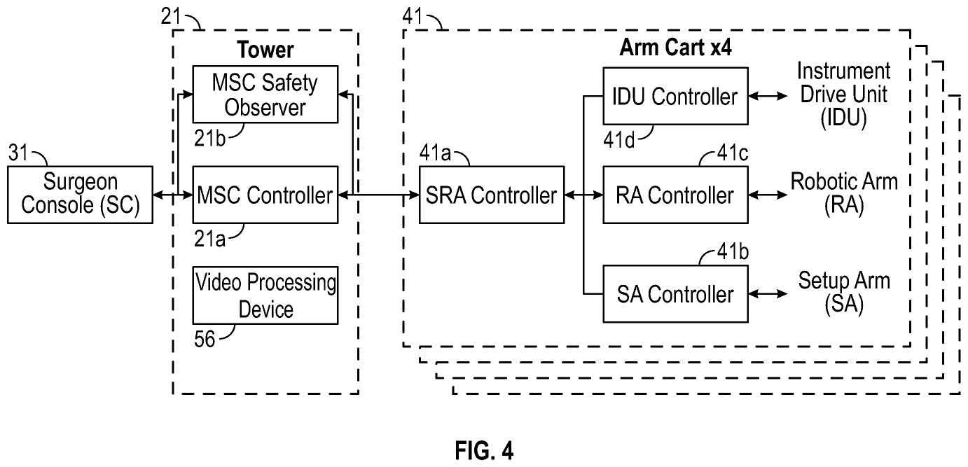

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand-eye transform function executed by the controller 21a. The hand-eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The video processing device 56 is configured to process the video feed from the endoscope camera 51 and to output a processed video stream on the first displays 32 of the surgical console 30 and/or the display 23 of the control tower 20.

FIG. 5 shows a flow chart illustrating the various operations of an exemplary method for clinical workspace simulation. Persons skilled in the art will appreciate that one or more operations of the method 500 may be performed in a different order, repeated, and/or omitted without departing from the scope of the disclosure. The operations of method 500 will be described with respect to a controller, e.g., controller 602 (FIG. 1) of virtual reality headset 600 (FIG. 1), but it will be understood that the illustrated operations are applicable to other systems and components thereof as well, such any suitable display, e.g., monitor. In various aspects, the illustrated method 500 can operate in controller 602 (FIG. 1), in a remote device, or in another server or system. Other variations are contemplated to be within the scope of the disclosure.

The disclosed method has the benefit of streamlining the surgical process by providing relevant information in advance of a patient's arrival, such as which components of the surgical robotic system 10 are to be used and how best to configure the components prior to the operation.

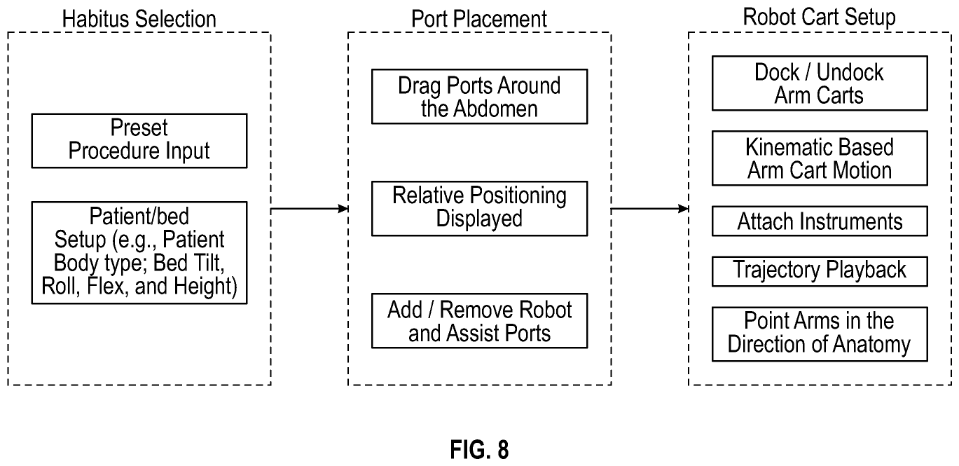

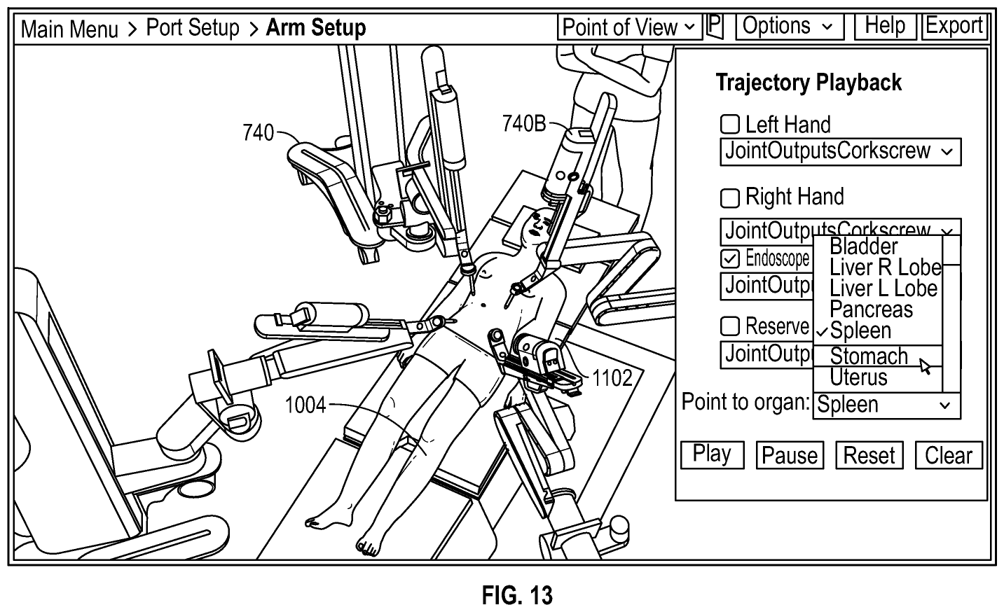

Initially, at step 502, the controller 602 accesses a surgical parameter from one or more robotic surgical operations by a sensor. The sensor may be an imaging device such as endoscopic camera 51 or another imaging device, e.g., on a mobile device or tablet. The surgical parameter, for example, may include a patient habitus (e.g., related to the mass, height, and/or positioning of the patient/anatomy and/or operating table), a virtual surgical port 1002 location in a virtual patient 1004 (FIG. 13) of the robotic surgical operation (e.g., considers optimal entry points into the virtual patient 1004 for a given procedure), and/or a robotic arm placement relative to the patient of the robotic surgical operation (e.g., considers optimal locations to avoid collision between independently moving surgical arms, given the target anatomy, bedside assist location, and other equipment in the room). The surgical parameter may also include positioning of the operating table, which may include the bed tilt, roll, flex, and or height (FIG. 8). The surgical parameter may further be entered by a user. The surgical parameter may further include instrumentation, trajectories, and/or endoscopic video. The user may adjust surgical parameters using a setup screen displayed on a display 608. The surgical parameter(s) may include surgical parameters of prior robotic surgical operations. The surgical parameter(s) of the prior robotic surgery may include a gender-based body habitus (such as a 5th percentile female), a number of ports and their location (such as five surgical ports were located on the patient's abdomen), and a number of virtual objects (such as the operation used four surgical robotic arms and a control tower).

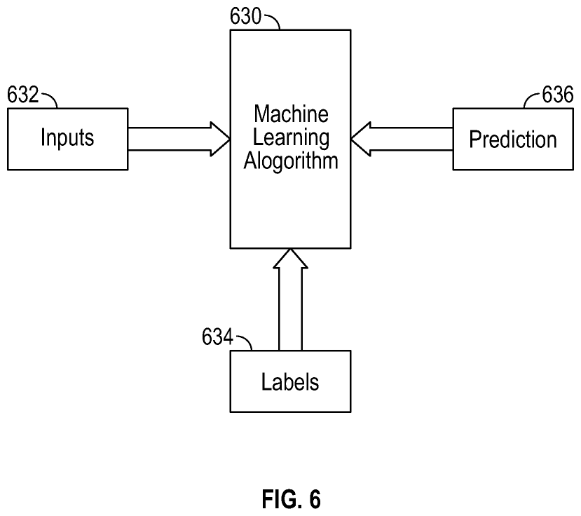

Next, at step 504, the controller 602 determines an optimized surgical parameter based on the captured surgical parameter. The optimized surgical parameter may be determined, for example, by using a machine learning network (FIG. 6) and/or by a set of rules. The optimized surgical parameter may include, for example, an optimized virtual surgical port 1002 placement location in a virtual patient and/or an optimized virtual object (e.g., robotic arm 40) placement location relative to the virtual patient 1004. The controller 602 may display a setup screen that enables the user to recall procedure settings, select a procedure, select a surgical table position (e.g., height, tilt, and/or roll), and/or a body habitus of a virtual patient (e.g., 5th percent female, 50th percent female, 95th percentile male).

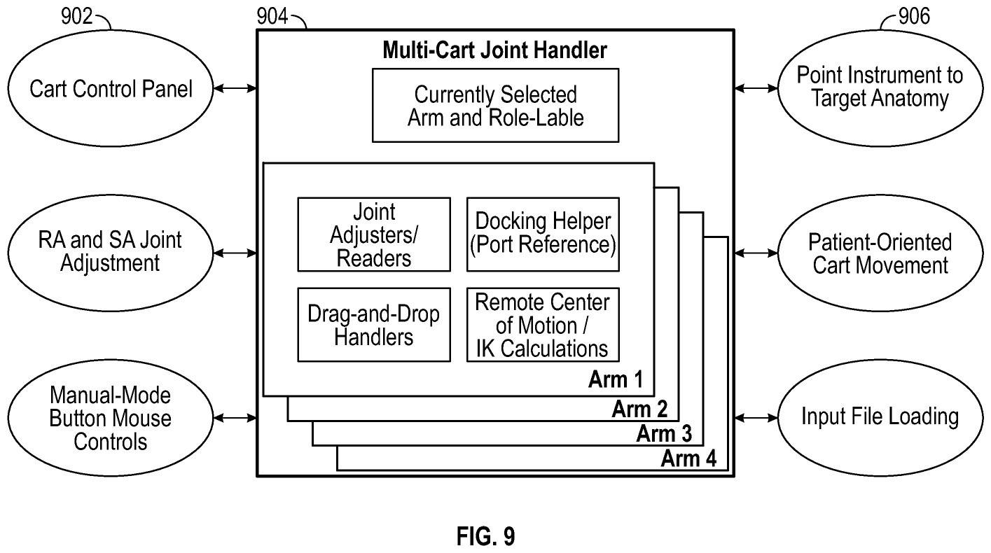

In aspects, the controller 602 may display a virtual robotic cart control panel 902 for adjusting the configuration of virtual the robotic arms (FIG. 9). The control panel 902 may enable the user to adjust 904 joint(s) of the virtual robotic arm, and/or drag and drop the virtual robotic arm at any location in the virtual operating room. The user may attach a surgical instrument to the virtual robotic arm. Each virtual object 740 may be “docked” and/or “undocked” to disable/enable movement of the virtual object 740 within the virtual operating room 1100.

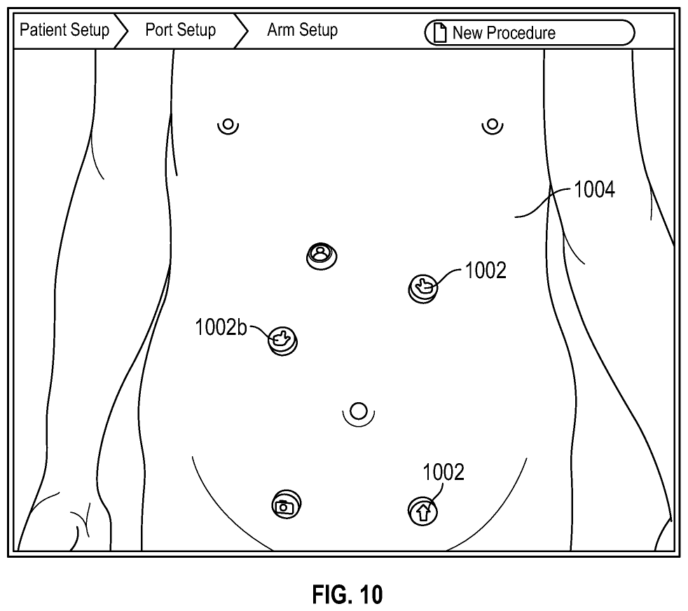

In aspects, the virtual surgical port 1002 may be displayed as a placeholder spherical port 1002b (FIG. 10). A ray-casting method may be used to find a terrain of the patient's abdomen. For example, a line may be projected from each spherical port in a direction of the orientation of the surgical table 1102. The spherical ports 1002b may be clicked and dragged (FIG. 8) around contours of the abdomen (FIG. 10).

In aspects, if a virtual surgical port 1002 gets too close to another virtual surgical port 1002 and/or approaches an outer bound of the abdomen, the controller 602 notifies the user, for example, by an alarm such as a sound and/or a visual warning (such as text and/or turning the surgical port red). The coordinates of a surgical port may be displayed on the display 608.

Next, at step 506, the controller 602 generates a simulated patient, including a simulated patient habitus based on a user input and/or a patient medical record. In aspects, the controller 602 may receive a user input for modifying the simulated patient habitus and modify the simulated patient habitus based on the user input. The user input may include sliders or other inputs to adjust patient habitus dimensions, body positions, and/or leg/arm positions.

Next, at step 508, the controller 602 generates a setup guide based on the generated simulated patient habitus and/or the determined optimized surgical parameter. The setup guide may include the optimized surgical port placement location and/or the optimized robotic arm 40 placement location. The setup guide may represent a clinical workspace simulation, which may be used, for example, to guide staff in setting up a surgical robotic system 10. When generating a setup guide, at step 508, the controller 602 may render one or more virtual objects (e.g., a virtual robot arm and/or a virtual surgical console) relative to a surgical table 1102 in the real-world environment. The initial position of the virtual object 740 may be based on, for example, the optimized surgical parameter, a type of surgical procedure, a patient body habitus, objects in the virtual operating room (e.g., the surgical table 1102 and/or clinical staff), and/or a surgical port location. The virtual object 740 may move and function analogously to its real-world counterpart so that a user (e.g., a clinician) may position and move the virtual object 740 and/or the virtual object's components to determine an initial setup for a surgery. The virtual surgical ports may be further located based on different patient anatomy. For example, a virtual surgical port may be placed directly above the belly button, the liver, and/or other anatomical features.

In aspects, the controller 602 may receive a user input for modifying the surgical ports based on the user input. For example, the user may drag the surgical ports around the abdomen of the virtual patient, and/or add or remove surgical ports. The controller 602 may enable the saving and recalling of previous surgical port positions. The relative positioning of the virtual surgical ports may be displayed on the display.

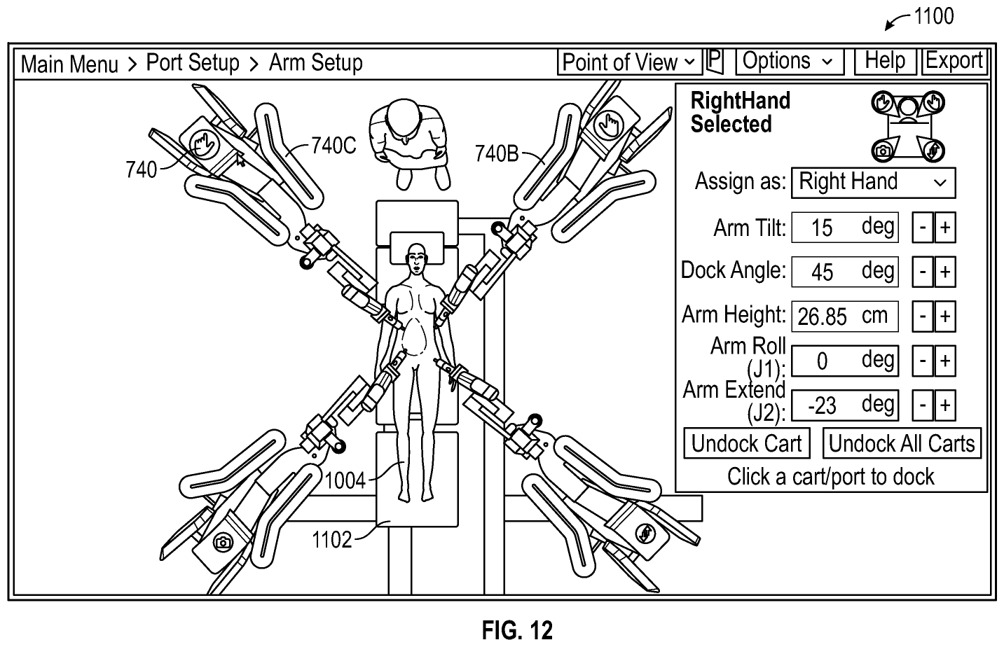

In aspects, the controller 602 may generate a revised surgical port placement location in a trunk segment of the simulated patient. In aspects, the controller 602 may generate a revised optimized robotic arm placement location 740c relative to the virtual patient 1104 (FIG. 12). The optimized robotic arm placement location and the revised surgical port placement location may be further based on the simulated patient habitus. Revised surgical port placement may be based on the type of procedure being performed. Thus, an improperly placed port would be moved to a region corresponding to the procedure being performed (e.g., from thoracic cavity to abdominal cavity).

Next, at step 510, the controller 602 displays the setup guide on a display 608 of the virtual reality headset 600, which may include a 3-D representation of several virtual robotic arms positioned around a virtual surgical table 1102 as shown in FIG. 11. The controller 602 may also suggest further optimal placement of these virtual objects 740, 740b based on their function and the type of surgery. In aspects, the controller 602 may display the setup guide on a user device, such as a mobile device and/or a tablet.

In aspects, the controller 602 may receive an indicated anatomy from a user. The optimized robotic arm placement location 740c (FIG. 12) may be further based on targeting the indicated anatomy.

In aspects, the controller 602 may receive input from more than one user. For example, one user can manipulate robotic arm positions on a laptop, while a second user is wearing the virtual reality headset 600 viewing the system 10 from the assistant's field-of-view. Similarly, the setup guide configuration can be exported to a virtual reality headset 600 where users can visualize virtual surgical components overlaid on a virtual image of an operating room, serving as a setup guide for the indicated procedure.

In aspects, the controller 602 may detect a potential collision between virtual objects 740, 740b. The controller 602 may use a bounding volume technique (such as axis-aligned bounding boxes or by bounding spheres) to detect collisions between virtual objects (such as a robotic arm, the virtual surgical table 1102, a control tower, and/or a surgical console) in the setup guide. In aspects, the virtual objects 740, 740b may be assigned 3-D coordinates in the setup guide to help with detecting collisions.

The controller 602 may display, on the display 608, an indication to a user providing a suggestion on avoiding the potential collision based on the determined collision. The controller 602 may also display an indication to move the second virtual object 740b by a predetermined distance or to the other side of the virtual surgical table 1102. Although two virtual objects are used in the above example, multiple objects may be rendered. The controller 602 may further display information on what further actions are blocked by an ongoing collision and suggestions on how to resolve the potential collisions from the further actions.

The controller 602 may provide enhanced feedback to the clinical staff by overlaying information on the setup guide, such as recommended surgical port entry points 1002c on the virtual patient's abdomen and/or the positioning of virtual objects (such as virtual robotic arms and/or virtual surgical console, (see FIG. 15) around the virtual surgical table 1102). In aspects, the controller 602 may render real-time measurements or suggestions of the surgical port entry points 1002c based on the patient body habitus for different locations on the virtual patient. For example, the controller 602 may display an indication that the virtual surgical port 1002 should be about 5 cm above and about 5 cm to the left of the naval.

In aspects, the controller 602 may render a visual overlay on the patient and/or the virtual robotic arm. The controller 602 may render a visual overlay such as a possible collision warning and/or a suggested surgical port entry point 1002c placement on a patient 1004 (FIG. 11).

The controller 602 may render the virtual surgical port 1002 entry point 1002c in the abdominal portion of the displayed virtual patient 1004 and display the rendered entry point on the display 608. In aspects, the controller 602 may generate an optimized virtual robotic arm placement location 740c (and or orientation) based on the virtual surgical port 1002 entry point 1102b (FIG. 11). The controller 602 may generate an initial virtual robotic arm placement based on the virtual surgical port 1002 entry points 1002c, then analyze the initial virtual robotic arm placement for possible collisions during surgery. If a potential collision is detected, the controller 602 may provide a series of corrective steps for adjusting placement of the virtual robotic arms (or other virtual object 740) which may be displayed on the display 608. The controller 602 can provide alternative placements of the virtual robotic arms (or other virtual object 740) and/or surgical port headset based on the potential collisions. Furthermore, the controller 602 is configured to automatically adjust the position and/or orientation of the virtual surgical port 1002 and/or virtual robotic arm (or other virtual object 740) based on the potential collision. In aspects, the controller 602 may overlay patient specific medical imaging, such as images from an MRI and/or a CAT scan. In aspects, the controller may generate an optimized virtual object 740 (e.g., robotic arm) location, orientation, and/or joint angles based on an organ or body part of interest.

In aspects, the controller 602 may provide an alarm (audio and/or visual) if a user deviates from normal procedures and may recommend a corrective course of action and how to follow the recommended course of action. In particular, the controller 602 may provide a series of corrective steps, which may be displayed on the display 608. The controller 602 can provide guidance on how to handle different situations that may block the user's progress when setting up and configuring the surgical robotic system 10.

The disclosed technology enables the user to brainstorm new setup ideas without needing a physical robotic surgical system and/or a human cadaver. The disclosed technology enables a realistic representation of system dynamics to better understand the impacts of troubleshooting and/or adjusting setups.

With reference to FIG. 6, the controller 200 may include a machine-learning algorithm 600 configured to make these evaluations. For example, the controller 200 may use machine learning to classify images. For example, machine learning may include a convolutional neural network (CNN) and/or a support vector machine (SVM). The CNN may be trained on previous data, for example, images of objects such as previous patients and/or surgical data, such as the type of operations and the specific robotic surgical equipment used for the operation.

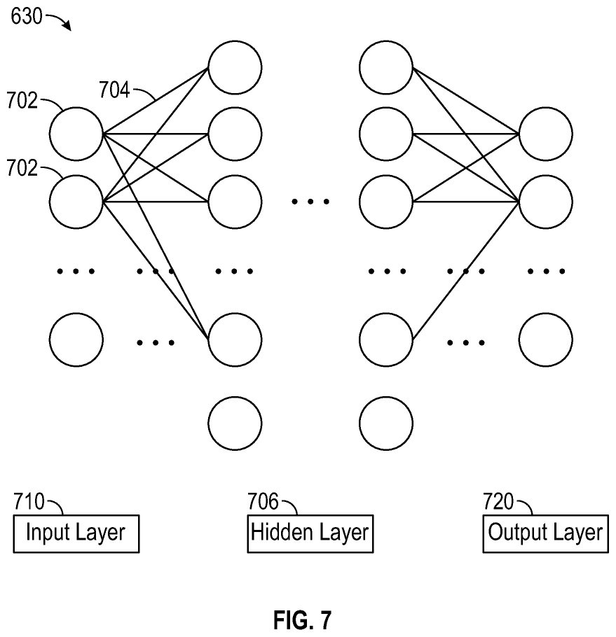

Referring to FIG. 7, generally, the machine learning network 600 (e.g., a convolutional deep learning neural network) of FIG. 6 includes at least one input layer 710, a plurality of hidden layers 706, and at least one output layer 720. The input layer 710, the plurality of hidden layers 706, and the output layer 720 all include neurons 702 (e.g., nodes). The neurons 702 between the various layers are interconnected via weights 674. Each neuron 702 in the machine learning network 600 computes an output value by applying a specific function to the input values coming from the previous layer. The function that is applied to the input values is determined by a vector of weights 704 and a bias. Learning, in the deep learning neural network, progresses by making iterative adjustments to these biases and weights. The vector of weights 704 and the bias are called filters (e.g., kernels) and represent particular features of the input (e.g., a particular shape). The machine learning network 600 may output logits.

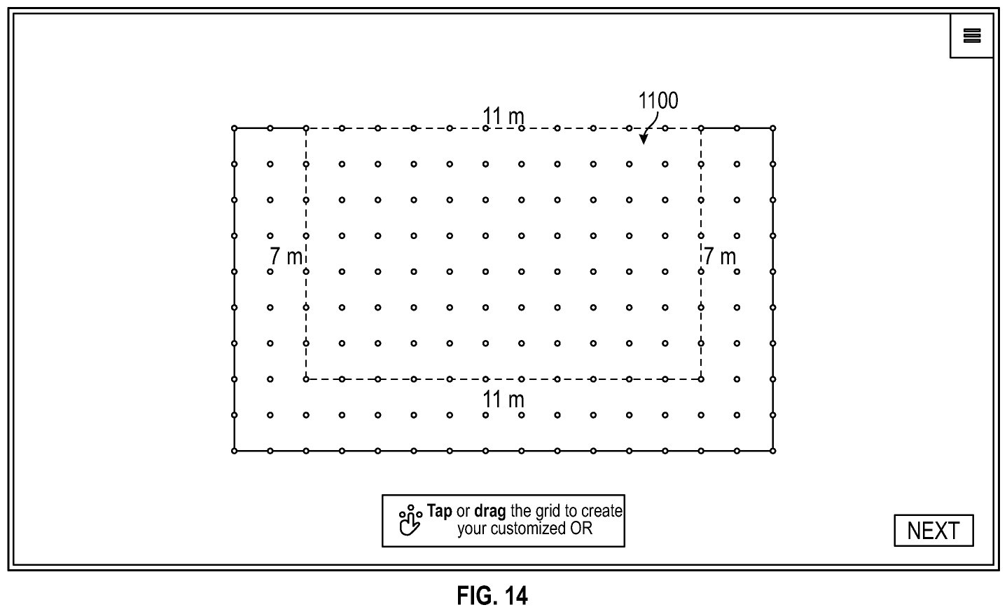

Referring to FIGS. 14 and 15, images of an image of a virtual operating room design screen and virtual robotic arms placed relative to a virtual surgical table 1102 in the virtual operating room as displayed on a display of the mobile device 610 (FIG. 1) are shown. The virtual operating room may be displayed on any device having a display, such as the mobile device 610, the virtual reality headset 600 (FIG. 1), or any other computing device.

In aspects, the controller 602 may receive an input from a user configuring dimensions and shape of an area for a virtual operating room (FIG. 14). For example, a user may indicate that the operating room is 11 meters by 7 meters square. In another example, the user may indicate that the virtual operating room may be “L” shaped. The operating room may be based on a real-world operating room, for example, the user may import a CAD file of the operating room.

In aspects, the controller 602 may receive an input from the user selecting virtual object(s). The virtual object(s) may include, for example, virtual robotic arm(s), control tower(s) 20, and/or a virtual surgical console, as well as subcomponents of the virtual robotic arm(s) and/or a virtual surgical console, such as a surgical tool and/or a generator.

In aspects, the controller 602 may render in 2D or 3D the virtual operating room based on the received input indicating the area for the virtual operating room. In aspects, the controller 602 may render (in 2D or 3D) the virtual object(s) relative to a virtual surgical table 1102 in the virtual operating room (FIG. 15).

In aspects, the controller 602 may display on a display 608 of a mobile device and/or a virtual reality headset the rendered virtual operating room and at least one virtual object relative to a virtual surgical table 1102 in the virtual operating room. The controller 602 may display an indication on the display to provide tips or hints to a user regarding functions. For example, the controller 602 may display a text box stating, “Tap one of the marked floor locations to move around the OR.” In aspects, the user may tap on one or more of the virtual objects to move them around the virtual operating room. The user may provide input to change the viewpoint or perspective of the displayed virtual operating room. The user may also change the view to an isometric view from a side view. The viewpoint/perspective may be locked by the user or by controller 602.

The virtual objects 740, 740b may include animations that may demonstrate the movements that the virtual object is capable of. For example, a virtual robotic arm may animate through a sequence of movements that simulate a specific surgical procedure that is indicated by the user and/or by the controller 602, including movement of control handles, robotic arm, and instrument.

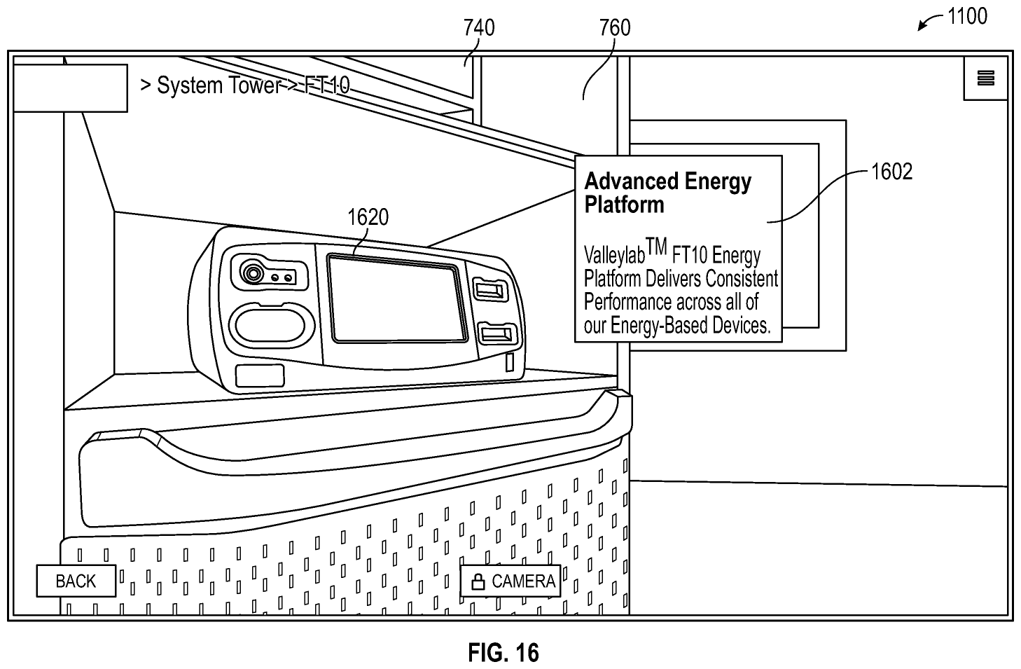

Regarding FIG. 16, an image of an electrosurgical generator 1620 of a virtual control tower 760 of the virtual operating room 1100 of FIG. 15 is shown. In aspects, a user may click on any of the virtual objects 740 to interact with the virtual objects 740. For example, a user may click on a virtual control tower 760, and the controller 602 may display on the display 608 a subcomponent, such as a generator 1620 and an information window 1602 regarding the subcomponent. For example, the information window 1602 may display information such as model numbers, specifications, and/or other details regarding the subcomponent.

In aspects, a plurality of users may navigate the virtual operating room 1100 simultaneously and/or interact with any of the virtual objects 740 using virtual reality headset 600 (FIG. 1). The disclosed technology enables the one or more users to collaborate whether they are located local or remote. Each of the users may have a unique avatar configured for interaction with the other users in the virtual operating room 1100. For example, two clinicians located in different physical regions or spaces may navigate the virtual operating room 1100 each using using virtual reality headset 600 (FIG. 1) to plan for an upcoming surgical procedure. The two clinicians, navigating in the virtual operating room 1100 may review the optimized virtual surgical port 1002 placement location in a virtual patient and/or the robotic arm 40 placement location relative to the virtual patient 1004 (FIG. 13).

It will be understood that various modifications may be made to the aspects disclosed herein. In aspects, the sensors may be disposed on any suitable portion of the robotic arm. Therefore, the above description should not be construed as limiting but merely as exemplifications of various aspects. Those skilled in the art will envision other modifications within the scope and spirit of the claims appended thereto.

What is claimed is:

1. A computer-implemented method for clinical workplace simulation, the method comprising:
receiving a first input from a user indicating an area for a virtual operating room;
receiving a second input from the user selecting at least one virtual object;
rendering the virtual operating room based on the received input indicating the area for the virtual operating room;
rendering the at least one virtual object relative to a virtual surgical table in the virtual operating room; and
displaying on a display the rendered virtual operating room and the at least one virtual object relative to a virtual surgical table in the virtual operating room.

2. The computer-implemented method of claim 1, wherein the virtual object includes at least one of a virtual robotic arm, a virtual surgical tower, or a virtual surgical console.

3. The computer-implemented method of claim 1, wherein the display includes a display of at least one of a mobile device, a computer, or a virtual reality headset.

4. The computer-implemented method of claim 1, wherein the displayed virtual operating room includes a viewpoint, wherein a viewpoint includes at least one of a perspective or a zoom level.

5. The computer-implemented method of claim 4, further comprising:
receiving a third input indicating a change in the viewpoint; and
displaying the changed viewpoint.

6. The computer-implemented method of claim 1, further comprising:
receiving a fourth input for interacting with the virtual object.

7. The computer-implemented method of claim 6, further comprising:
displaying a text window, indicating information relating to the virtual object.

8. The computer-implemented method of claim 6, further comprising:
displaying an animation of the virtual object.

9. The computer-implemented method of claim 1, further comprising:
generating a virtual patient including a simulated patient habitus based on at least one of a fifth input or a patient medical record.

10. The computer-implemented method of claim 1, further comprising:
receiving a sixth input placing the at least one virtual object at a virtual object placement location; and
rendering the at least one virtual object at the virtual object placement location.

11. The computer-implemented method of claim 10, further comprising:
receiving an indicated anatomy of the virtual patient,
wherein the virtual object placement location is further based on targeting the indicated anatomy.
